(19) United States
(12) Patent Application Publication
(10) Pub. No.: US 2026/0229331 A1
(43) Pub. Date: Aug. 6, 2026

(54) DIFFUSION-BASED PRIORS FOR INVERSE PROBLEMS FOR MEDICAL DECISION MAKING

(71) Applicant: NEC Laboratories America, Inc., Princeton, NJ (US)

(72) Inventors: Zachary Izzo, Cranbury, NJ (US); Haoxuan Chen, Stanford, CA (US)

(21) Appl. No.: 19/461,037

(22) Filed: Jan. 27, 2026

Related U.S. Application Data
(60) Provisional application No. 63/751,955, filed on Jan. 31, 2025.
(60) Provisional application No. 63/814,467, filed on May 30, 2025.

(51) Int. Cl. G16H 20/00; G16H 10/60; G16H 30/40; G16H 50/20

(52) U.S. Cl. CPC G16H 20/00; G16H 10/60; G16H 30/40; G16H 50/20

(57) ABSTRACT

Methods and systems include recovering an original object from a measured object that includes a distortion, including approximation-free sampling from a posterior distribution of the measured object using a diffusion model. An action is performed responsive to the recovered original object.

### RELATED APPLICATION INFORMATION

This application claims priority to U.S. Patent Application No. 63/751,955, filed on Jan. 31, 2025, and to U.S. Patent Application No. 63/814,467, filed on May 30, 2025, each incorporated herein by reference in its entirety.

### BACKGROUND

### Technical Field

The present invention relates to machine learning models and, more particularly, to diffusion models to solve inverse problems.

### Description of the Related Art

Inverse problems arise in a variety of contexts where an incomplete or noisy measurement is available and more detailed information is needed. For example, given a blurry picture or video of a scene, the inverse problem may be to remove the blur or other aberrations to recover the original image. Inverse problems are often ill-posed, such that there are many possible original objects which could have produced the observed measurement.

### SUMMARY

A method includes recovering an original object from a measured object that includes a distortion, including approximation-free sampling from a posterior distribution of the measured object using a diffusion model. An action is performed responsive to the recovered original object.

A system includes a hardware processor and a memory that stores a computer program. When executed by the hardware processor, the computer program causes the hardware processor to recover an original object from a measured object that includes a distortion, including approximation-free sampling from a posterior distribution of the measured object using a diffusion model, and to perform an action responsive to the recovered original object.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a set of images showing how a reconstruction of a blurred image can result in different outputs, with details differing between them, in accordance with an embodiment of the present invention;

FIG. 2 is a block/flow diagram of a method for sampling from a posterior distribution for object reconstruction, in accordance with an embodiment of the present invention;

FIG. 3 is pseudo-code of a method for resampling a distribution, in accordance with an embodiment of the present invention;

FIG. 4 is pseudo-code of a method for approximation-free diffusion posterior sampling via stochastic differential equations, in accordance with an embodiment of the present invention;

FIG. 5 is pseudo-code of a method for approximation-free diffusion posterior sampling via ordinary differential equations, in accordance with an embodiment of the present invention;

FIG. 6 is pseudo-code of a method for correcting samples to enhance exploration, in accordance with an embodiment of the present invention;

FIG. 7 is a block/flow diagram of a method of reconstructing an image, in accordance with an embodiment of the present invention;

FIG. 8 is a block diagram of a healthcare facility that reconstructs patient data for medical decision making, in accordance with an embodiment of the present invention;

FIG. 9 is a computing device that can perform object recovering, in accordance with an embodiment of the present invention;

FIG. 10 is a diagram of an exemplary neural network architecture that can be used to implement part of a diffusion model, in accordance with an embodiment of the present invention; and

FIG. 11 is a diagram of an exemplary deep neural network architecture that can be used to implement part of a diffusion model, in accordance with an embodiment of the present invention.

### DETAILED DESCRIPTION OF PREFERRED EMBODIMENTS

Bayesian posterior sampling may be used to select a possible solution to an inverse problem, sampling from the posterior distribution of an object of interest given a noisy or incomplete measurement of the object and some prior distribution. In this manner the most likely original object can be selected from among the many possibilities. Ensemble-based posterior sampling may be used with a diffusion-based prior without making heuristic approximations to an evolved posterior process.

The prior may be specified by a score-based diffusion model. Machine learning is used to train this diffusion model-a neural network that learns the statistical structure of natural images by observing large datasets of real photographs and illustrations. The model is trained to reverse a gradual noising process, thereby learning what natural images “look like” in terms of textures, shapes, colors, and spatial organization. At generation time, the model starts from random noise and iteratively denoises it to produce new images that follow the learned distribution of natural images. Mathematically, this model transforms a Gaussian distribution p₀ into an arbitrary target distribution p_{T} via a sequence of intermediate distributions pₜ as the time t ranges from 0 to T. The distribution encoded by the diffusion model can then be used to solve an inverse problem by considering the evolution of the corresponding posterior distributions qₜ, where qₜ is the posterior associated with the prior pₜ for any t. Using the exact dynamics of the posterior provides improved accuracy.

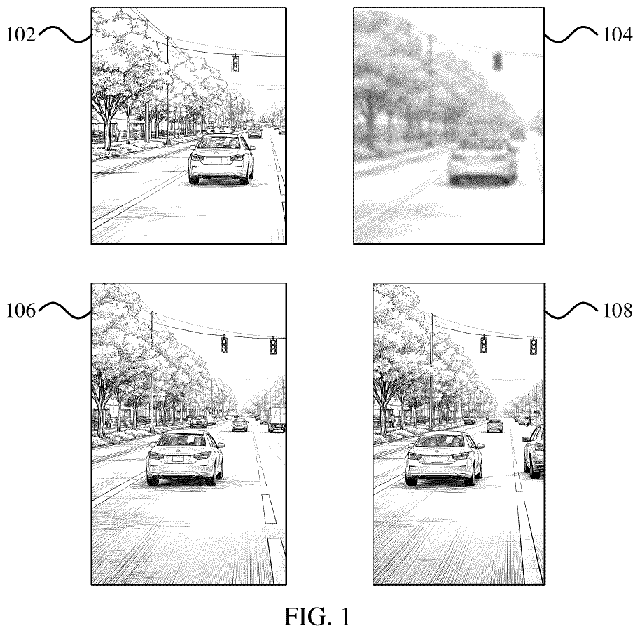

Referring now to FIG. 1, an original image 102 is shown as a road scene. This reflects the actual scene. A blurred image 104 shows a measurement of that same scene, but in this view the image is blurred. This may occur because of weather conditions, problems with the camera's hardware, blurring software, or any of a host of other causes.

Recovered images 106 and 108 show two different attempts to remove the blurring from the blurred image 104 to recover the original image 102. This is an inverse problem, attempting to reverse whatever transformation caused the blurring to recover lost information. However, because details are lost in the blurring, attempts to recover of those details may produce inconsistent results, such that the recovered images 106 and 108 differ from one another and from the original image 102, in some cases omitting details that would have been present in the original image 102 and in other cases adding details that would have been absent in the original image 102.

Given a noisy or incomplete observation y of ground truth object x, the present embodiments may sample from the posterior distribution over x where the prior is specified by a diffusion model. Following the example above, the incomplete observation y may be represented by the blurred image 104 and the ground truth object x may be represented by the original image 102.

A trained diffusion model may be specified by the continuous-time score function s(x, t):=∇ₓ log pₜ(x). The likelihood of the observation y given the ground truth x is denoted as p(y|x). Given that the observation y is fixed, then μ_{y}(x)=−log p(y|x) is the negative log-likelihood function for a fixed observation y. In particular, with a prior p over solutions, the posterior q satisfies q(x)∝p(x)e^{-μ(x)}. The inverse likelihood may be defined as f(x)=e^{μ(x)} and Δf(x) is the Laplacian of f at x. The term V(x) is defined as

$$\frac{1}{2} {‖ x ‖}^{2}$$

and the quantity G(x, t) is defined as 2s(x, t)−2∇μ(x)+∇V(x). Training the diffusion model is mathematically equivalent to learning the score function s(x, t).

An ensemble of N particles (indexed by i) may be tracked across K discrete time steps (indexed by k) with time discretization step Δt so that the k^{th} discrete step corresponds to kΔt in continuous time. The i^{th} particle's value at the k^{th} time step may be represented by

$${x}_{i}^{k} .$$

The relationship between x and y may be expressed as y= [symbol] (x)+n, where x∈ [symbol] y∈ [symbol] and where [symbol] : [symbol] is a differentiable forward operator, linear or non-linear. The n∈ [symbol] represents measurement noise. Under a Bayesian framework, the posterior distribution to sample from is:

$$p ( x \vert y ) ∝ {p}_{0} ( x ) p ( y \vert x ) = {p}_{0} ( x ) {e}^{- {μ}_{y} ( x )}$$

where p₀(x) is a prior distribution.

A framework for modeling the prior begins with a forward diffusion process governed by the stochastic differential equation (SDE):

$$d {x}_{s} = F ( s ) {x}_{s} ds + G ( s ) d {w}_{s}$$

where (wₛ)_{s≥0} is Brownian motion and pₛ denotes the distribution of xₛ, with p₀ being the prior distribution. The corresponding reverse-time SDE is:

$$d {\overleftarrow{x}}_{t} = [ - F ( t ) {\overleftarrow{x}}_{t} ← + \frac{{G ( t )}^{2} + {V ( t )}^{2}}{2} {∇}_{x} log {\overleftarrow{p}}_{t} ( {\overleftarrow{x}}_{t} ) ] dt + V ( t ) d {w}_{t} ,$$

where [symbol] =p_{T}, [symbol] =p₀, [symbol] denotes _{*T-t}, and V: [symbol] is a scalar-valued function. The score function ∇ log [symbol] (x) is typically approximated by a neural network φ_{θ}(x, t) trained via score matching. The terms [symbol] and [symbol] denote the particle trajectory and its distribution when using the approximated score function φ_{θ}(x, t), with [symbol] being exactly Gaussian and [symbol] approximating the target distribution p₀.

The drift coefficient F(t) and diffusion coefficient G(t) may be reparameterized using:

$$s ( t ) := exp ( {\int }_{0}^{t} F ( ξ ) d ξ ) and σ ( t ) := \sqrt{{\int }_{0}^{t} \frac{{G ( ξ )}^{2}}{{s ( ξ )}^{2}} d ξ}$$

yielding

$$F ( t ) = \frac{\dot{s} ( t )}{s ( t )} and G ( t ) = s ( t ) \sqrt{2 \dot{σ} ( t ) σ ( t )} .$$

This reparameterization enables more accurate score estimation under appropriate choices of s and σ. This further allows for different implementations based on the choice of diffusion coefficient V. Setting V(t)=G(t)=s(t)\sqrt{2\dot{σ}(t)σ(t)} yields the SDE implementation:

$$d {\hat{\overleftarrow{x}}}_{t} = [ - \frac{\dot{s} ( t )}{s ( t )} {\hat{\overleftarrow{x}}}_{t} + 2 {s ( t )}^{2} \dot{σ} ( t ) σ ( t ) {ϕ}_{θ} ( {\hat{\overleftarrow{x}}}_{t} , t ) ] dt + s ( t ) \sqrt{2 \dot{σ} ( t ) σ ( t )} d {w}_{t}$$

Alternatively, setting V(t)=0 yields the probability-flow ODE (PF-ODE) implementation:

$$d {\hat{\overleftarrow{x}}}_{t} = [ - \frac{\dot{s} ( t )}{s ( t )} {\hat{\overleftarrow{x}}}_{t} + {s ( t )}^{2} \dot{σ} ( t ) σ ( t ) {ϕ}_{θ} ( {\hat{\overleftarrow{x}}}_{t} , t ) ] dt$$

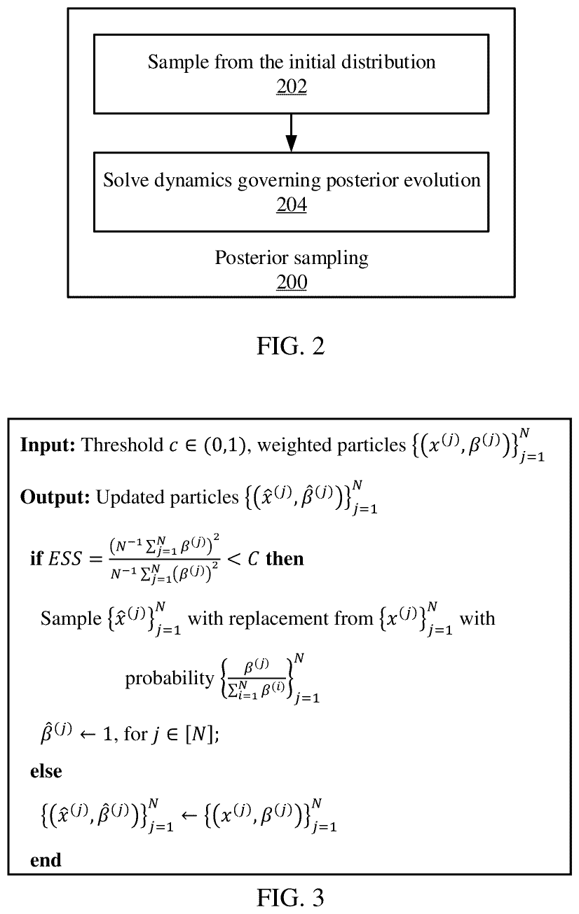

Referring now to FIG. 2, a method for posterior sampling 200 is shown. Posterior sampling 200 can be interpreted as solving a high-dimensional partial differential equation (PDE) that governs posterior distribution evolution using either the (stochastic) weighted particle method or the sequential Monte Carlo (SMC) method. The log-likelihood function μ_{y}(x) may be assumed to be at least twice differentiable with respect to x for fixed y. Assuming the prior distribution p(x) is represented by a diffusion model, with p₀(x) being approximated by [symbol] (x), obtained by simulation from a Gaussian [symbol] , the time-dependent posterior distribution may be defined as:

$${\hat{q}}_{y} ( x , t ) := \frac{{\hat{\overleftarrow{p}}}_{t} ( x ) {e}^{- {μ}_{y} ( x )}}{{\int }_{{ℝ}^{n}} {\hat{\overleftarrow{p}}}_{t} ( x ) {e}^{- {μ}_{y} ( x )} d x} : = \frac{{\hat{Q}}_{y} ( x , t )}{{\hat{Z}}_{y} ( t )} ,$$

where \hat{Q}_{y}(x, t)= [symbol] (x)e^{-μ}^{_{y}}^{(x)} is the unnormalized posterior, and \hat{Z}_{y}(t)= [symbol] \hat{Q}_{y}(x, t)dx is the normalizing constant.

Posterior sampling 200 includes sampling 202 from the initial distribution \hat{q}_{y}(x, 0) and solving 204 the PDE dynamics governing posterior evolution. Sampling 202 begins by sampling from \hat{q}_{y}(x, 0)∝ [symbol] (x)e^{-μ}^{_{y}}^{(x)}. Given differentiable μ_{y}(x), gradient-based samplers can be used. For linear Bayesian inverse problems with Gaussian noise, where [symbol] :=A∈ [symbol] and n~ [symbol] (0, Σ), assuming [symbol] = [symbol] (0, ρ²In), the initial distribution simplifies to:

$${\hat{q}}_{y} ( x , 0 ) ∝ exp ( - {( y - Ax )}^{T} {\sum }^{- 1} ( y - A x ) - \frac{1}{2 {ρ}^{2}} {‖ x ‖}_{2}^{2} ) = 𝒩 ( γ , {Λ}^{- 1} )$$

where

$$Λ = {A}^{T} {\sum }^{- 1} A + \frac{1}{{ρ}^{2}} {I}_{n} and γ = {Λ}^{- 1} {A}^{T} {\sum }^{- 1} y ,$$

Σ is the covariance matrix of the measurement noise, and ρ is the variance of the Gaussian distribution sampled from to use the base diffusion model. Exemplary values may be ρ=1 and Σ=ϵ²In for some small value of ϵ, such as ϵ=0.2.

PDE dynamics (\hat{Q}_{y}(x, t))_{t∈[0,T]} are derived based on the diffusion process from ( [symbol] )_{t∈[0,T]}. These dynamics are normalized to yield the PDE that evolves (\hat{q}_{y}(x, t))_{t∈[0,T]}. The Fokker-Planck equation evolving from [symbol] to [symbol] is:

$$\frac{\partial }{\partial t} {\hat{\overleftarrow{p}}}_{t} = - {∇}_{x} \cdot ( ( - F ( t ) x + \frac{{G ( t )}^{2} + {V ( t )}^{2}}{2} {ϕ}_{θ} ( x , t ) ) {\hat{\overleftarrow{p}}}_{t} ) + \frac{1}{2} {V ( t )}^{2} {Δ}_{x} {\hat{\overleftarrow{p}}}_{t} .$$

Substituting [symbol] (x)=\hat{Q}_{y}(x, t)exp(μ_{y}) yields:

$$\frac{\partial }{\partial t} {\hat{Q}}_{y} = - {∇}_{x} \cdot ( ( \hat{H} ( x , t ) - {V ( t )}^{2} {∇}_{x} {μ}_{y} ) {\hat{Q}}_{y} ) + \frac{1}{2} {V ( t )}^{2} {Δ}_{x} {\hat{Q}}_{y} + ( \frac{1}{2} {V ( t )}^{2} ( {‖ {∇}_{x} {μ}_{y} ‖}_{2}^{2} - {Δ}_{x} {μ}_{y} ) - {\hat{H} ( x , t )}^{T} {∇}_{x} {μ}_{y} ) {\hat{Q}}_{y}$$

where

$$\hat{H} ( x , t ) := - F ( t ) x + \frac{{G ( t )}^{2} + {V ( t )}^{2}}{2} {ϕ}_{θ} ( x , t )$$

is the original drift.

### Defining

$$U ( x , t ) := \frac{1}{2} {V ( t )}^{2} ( {‖ {∇}_{x} {μ}_{y} ‖}_{2}^{2} - {Δ}_{x} {μ}_{y} ) ,$$

the following PDE for \hat{q}_{y}(x, t) is:

$$\frac{\partial }{\partial t} {\hat{q}}_{y} = - {∇}_{x} \cdot ( ( \hat{H} ( x , t ) - {V ( t )}^{2} {∇}_{x} {μ}_{y} ) {\hat{q}}_{y} ) + \frac{1}{2} {V ( t )}^{2} {Δ}_{x} {\hat{q}}_{y} + ( U ( x , t ) - {\hat{H} ( x , t )}^{T} {∇}_{x} {μ}_{y} - {\int }_{{ℝ}^{n}} ( U ( x , t ) - {\hat{H} ( x , t )}^{T} {∇}_{x} {μ}_{y} ) {\hat{q}}_{y} d x ) {\hat{q}}_{y}$$

Two ensemble-based posterior samplers are described herein within the SMC framework, which can also be interpreted as solving the PDE numerically via (stochastic) weighted particles. The posterior evolution can be simulated via the following dynamics of a single weighted particle (xₜ, βₜ):

$$\{ \begin{matrix}d {x}_{t} = ( \hat{H} ( {x}_{t} , t ) - {V ( t )}^{2} {∇}_{x} {μ}_{y} ( {x}_{t} ) ) d t + V ( t ) d {w}_{t} , \\ d {β}_{t} = ( U ( {x}_{t} , t ) - {\hat{H} ( {x}_{t} , t )}^{T} {∇}_{x} {μ}_{y} ( {x}_{t} ) ) {β}_{t} dt - \\ ( {\int }_{{ℝ}^{n}} ( U ( x , t ) - {\hat{H} ( x , t )}^{T} {∇}_{x} {μ}_{y} ( x ) ) ( {P}_{β} {γ}_{t} ) ( x ) d x ) {β}_{t} dt\end{matrix} ,$$

where γₜ(x, β) denotes the joint probability distribution of (xₜ, βₜ) and P_{β}γₜ(x):=∫_{R} βγₜ(x, β)dβ denotes the weighted projection of γₜ onto x. To effectively approximate the integral in P_{β}γₜ, the empirical measure

$${γ}_{t} ( x , β ) \approx \frac{1}{N} {\sum }_{i = 1}^{N} {δ}_{( {x}_{t}^{( 1 )} , {β}_{t}^{( t )} )}$$

formed by N weighted particles can be used to approximate γₜ(x, β).

This leads to the following joint dynamics for

$${\{ ( {x}_{t}^{( i )} , {β}_{t}^{( i )} ) \}}_{i = 1}^{N} :$$

$$\{ \begin{matrix}d {x}_{t}^{( i )} = ( \hat{H} ( {x}_{t}^{( i )} , t ) - {V ( t )}^{2} {∇}_{x} {μ}_{y} ( {x}_{t}^{( i )} ) ) dt + V ( t ) d {w}_{t}^{( i )} , \\ d {β}_{t}^{( i )} = ( U ( {x}_{t}^{( i )} , t ) - {\hat{H} ( {x}_{t}^{( i )} , t )}^{T} {∇}_{x} {μ}_{y} ( {x}_{t}^{( i )} ) ) {β}_{t}^{( i )} dt - \\ ( \frac{1}{N} {\sum }_{j = 1}^{N} ( U ( {x}_{t}^{( j )} , t ) - {\hat{H} ( {x}_{t}^{( j )} , t )}^{T} {∇}_{x} {μ}_{y} ( {x}_{t}^{( j )} ) ) {β}_{t}^{( j )} ) {β}_{t}^{( i )} dt\end{matrix} ,$$

with initial conditions

$${x}_{0}^{( i )} ∼ {\hat{q}}_{y} ( \cdot , 0 ) and {β}_{0}^{( i )} = 1 ,$$

for i∈[N]. The weighted projection equals

$$\frac{1}{N} {β}_{t}^{( j )}$$

when

$$x = {x}_{t}^{( j )}$$

for some j, and zero otherwise.

While numerical discretization of the weighted particle dynamics yields a prototypical sampling algorithm, the particle weights

$${β}_{t}^{( i )}$$

may diverge during simulation, reducing the ensemble's Effective Sample Size (ESS). To address this, a resampling strategy may be applied. Such a resampling sub-routine essentially performs global moves by eliminating low-weight particles and duplicating high-weight ones, similar to a birth-death process. However, the resampling approach is computationally more efficient as the weight dynamics can be parallelized.

Referring now to FIG. 3, pseudo-code is provided that may be used for resampling when performing direct discretization in a diffusion model. An SDE implementation of a diffusion model may set V(t)=G(t)=s(t)\sqrt{2\dot{σ}(t)σ(t)}. Direct discretization may be performed with an Euler-Maruyama scheme and resampling may be performed as set out in FIG. 3 at the end of each iteration.

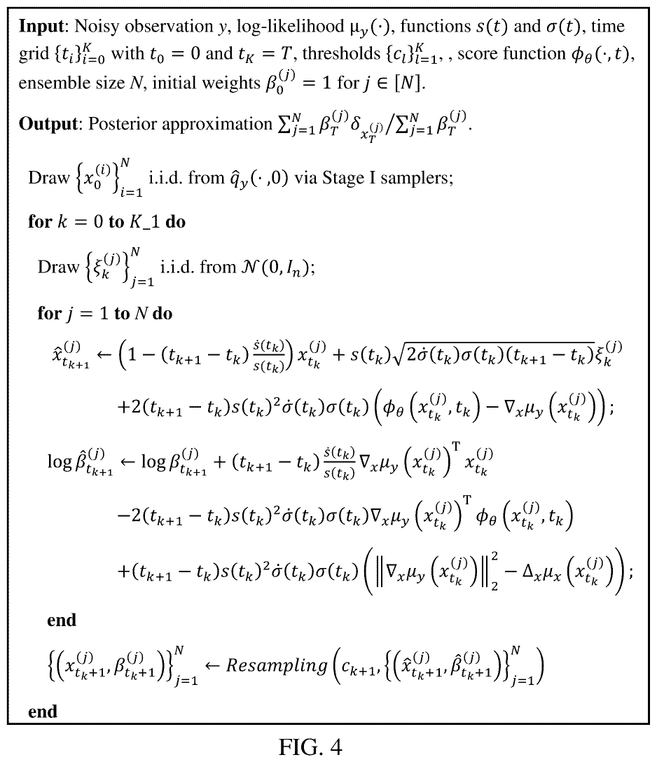

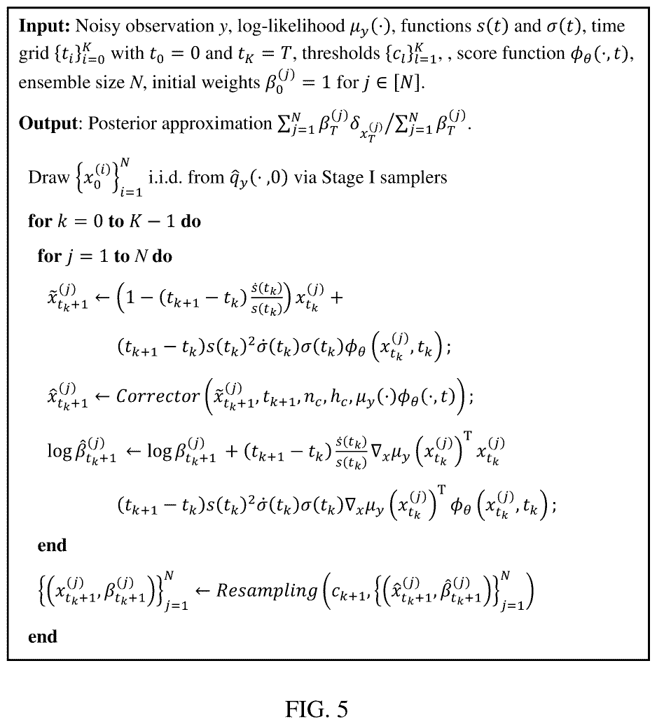

Referring now to FIG. 4, pseudo-code is provided that may be used for approximation-free diffusion posterior sampling via SDE. In practical implementations the update is the same for all particles and so cancels out when weights are normalized. This cancellation property also holds for ordinary differential equation (ODE) approaches as described in greater detail below. The computational cost can be reduced for high-dimensional problems by using smaller ensembles, omitting the resampling step, or by returning the particle with the highest weight as the best estimator.

Referring now to FIG. 5, pseudo-code is provided that may be used for an approximation-free diffusion posterior sampling method using ODE. This implementation is based on a probability flow ODE by setting V(t)=0. While this leads to ODE dynamics described above, relying solely on deterministic evolution may not sufficiently explore the target distribution. A stochastic corrector step, described below, may be used to enhance exploration.

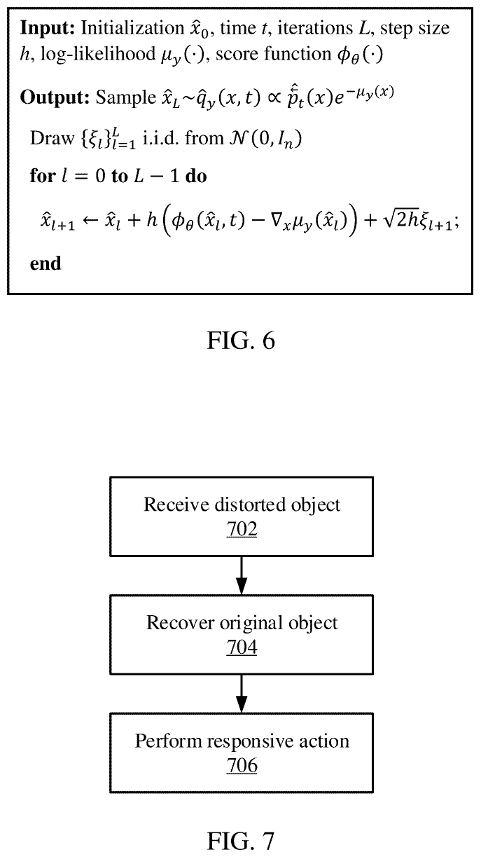

Referring now to FIG. 6, pseudo-code is provided that may be used for a corrector function. The corrector uses the unadjusted Langevin algorithm (ULA) to draw samples from the intermediate posterior distribution \hat{q}_{y}(x, t)∝ [symbol] (x)e^{-μ}^{_{y}}^{(x)} at each timestep. The probability flow ODE is discretized and both resampling and ULA correction are applied for adjustments.

Referring now to FIG. 7, a method of recovering an object, such as an image, is shown. Block 702 receives a distorted object, such as an image that has been blurred by the circumstances of its capture or intentionally by software. Block 704 recovers the original object as described above, for example by posterior sampling with weighted particles. Block 706 then performs an action responsive to the recovered object.

The sample drawn from the posterior is the recovered object and is not restricted to images. The exact same procedure could be applied to denoise observations from any type of sensor reading, provided that a diffusion model is trained on clean readings from that sensor.

For example, in instances where the object is an image captured from a video, such as in a security setting or for a self-driving vehicle, the image may be blurred or obscured by weather, objects, or other environmental conditions. Block 704 can then recover the original image, which can then be used to implement a security or self-driving action. In some cases the object may include medical data, such as information gathered from biometric sensors that may be noisy or faulty. The patient's true condition may be inferred in block 704, and the responsive action in block 706 may include a treatment action.

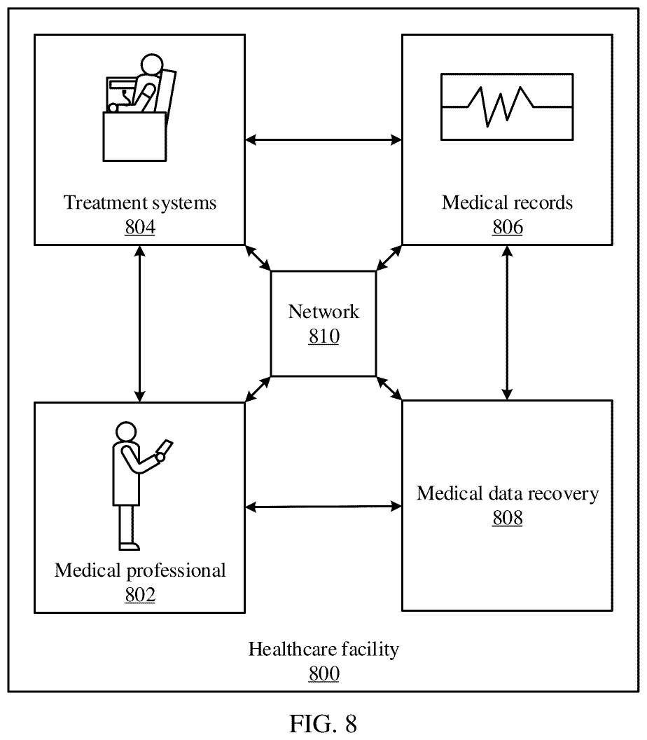

Referring now to FIG. 8, a diagram of time series analysis is shown in the context of a healthcare facility 800. Medical data recovery 808 may be used to enhance medical decision making by medical professionals 802 by correcting noisy data in a patient's medical records 806.

The healthcare facility may include one or more medical professionals 802 who review information extracted from a patient's medical records 806 to determine their healthcare and treatment needs. These medical records 806 may include self-reported information from the patient, test results, and notes by healthcare personnel made to the patient's file. Treatment systems 804 may furthermore monitor patient status to generate medical records 806 and may be designed to automatically administer and adjust treatments as needed.

The different elements of the healthcare facility 800 may communicate with one another via a network 810, for example using any appropriate wired or wireless communications protocol and medium. Thus medical data recovery 808 receives information about the patient from medical professionals 802 and from medical records 806 and identifies the patient's health condition by recovering their true state from the noise. The medical data recovery 808 may further work in tandem with treatment systems 804, for example by helping to identify the patient's true condition and to automatically alter or stop a treatment that would result in a negative health outcome.

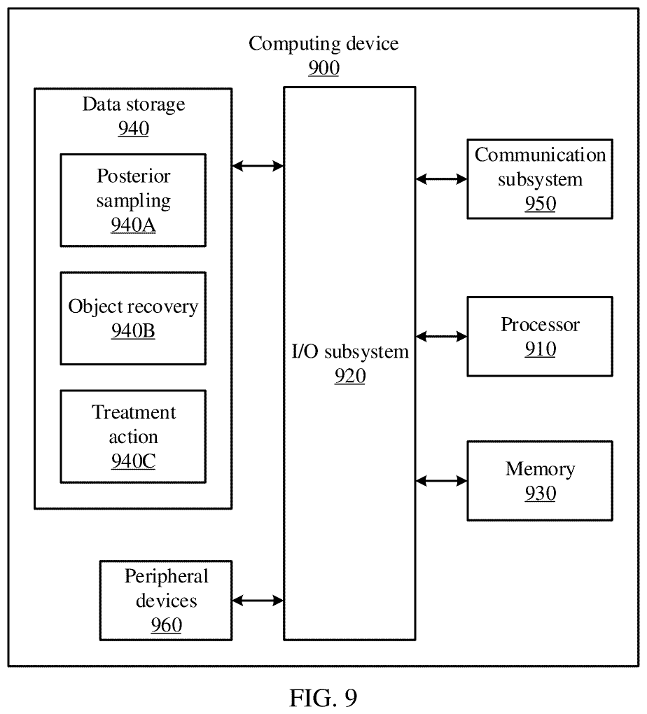

Referring now to FIG. 9, an exemplary computing device 900 is shown, in accordance with an embodiment of the present invention. The computing device 900 is configured to recover objects that have been distorted in some way.

The computing device 900 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 900 may be embodied as one or more compute sleds, memory sleds, or other racks, sleds, computing chassis, or other components of a physically disaggregated computing device.

As shown in FIG. 9, the computing device 900 illustratively includes the processor 910, an input/output subsystem 920, a memory 930, a data storage device 940, and a communication subsystem 950, and/or other components and devices commonly found in a server or similar computing device. The computing device 900 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 930, or portions thereof, may be incorporated in the processor 910 in some embodiments.

The processor 910 may be embodied as any type of processor capable of performing the functions described herein. The processor 910 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 930 may be embodied as any type of volatile or non-volatile memory or data storage capable of performing the functions described herein. In operation, the memory 930 may store various data and software used during operation of the computing device 900, such as operating systems, applications, programs, libraries, and drivers. The memory 930 is communicatively coupled to the processor 910 via the I/O subsystem 920, which may be embodied as circuitry and/or components to facilitate input/output operations with the processor 910, the memory 930, and other components of the computing device 900. For example, the I/O subsystem 920 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 920 may form a portion of a system-on-a-chip (SOC) and be incorporated, along with the processor 910, the memory 930, and other components of the computing device 900, on a single integrated circuit chip.

The data storage device 940 may be embodied as any type of device or devices configured for short-term or long-term storage of data such as, for example, memory devices and circuits, memory cards, hard disk drives, solid state drives, or other data storage devices. The data storage device 940 can store program code 940A for posterior sampling, 940B for object recovery, and/or 940C for performing treatment actions. Any or all of these program code blocks may be included in a given computing system. The communication subsystem 950 of the computing device 900 may be embodied as any network interface controller or other communication circuit, device, or collection thereof, capable of enabling communications between the computing device 900 and other remote devices over a network. The communication subsystem 950 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 900 may also include one or more peripheral devices 960. The peripheral devices 960 may include any number of additional input/output devices, interface devices, and/or other peripheral devices. For example, in some embodiments, the peripheral devices 960 may include a display, touch screen, graphics circuitry, keyboard, mouse, speaker system, microphone, network interface, and/or other input/output devices, interface devices, and/or peripheral devices.

Of course, the computing device 900 may also include other elements (not shown), as readily contemplated by one of skill in the art, as well as omit certain elements. For example, various other sensors, input devices, and/or output devices can be included in computing device 900, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 900 are readily contemplated by one of ordinary skill in the art given the teachings of the present invention provided herein.

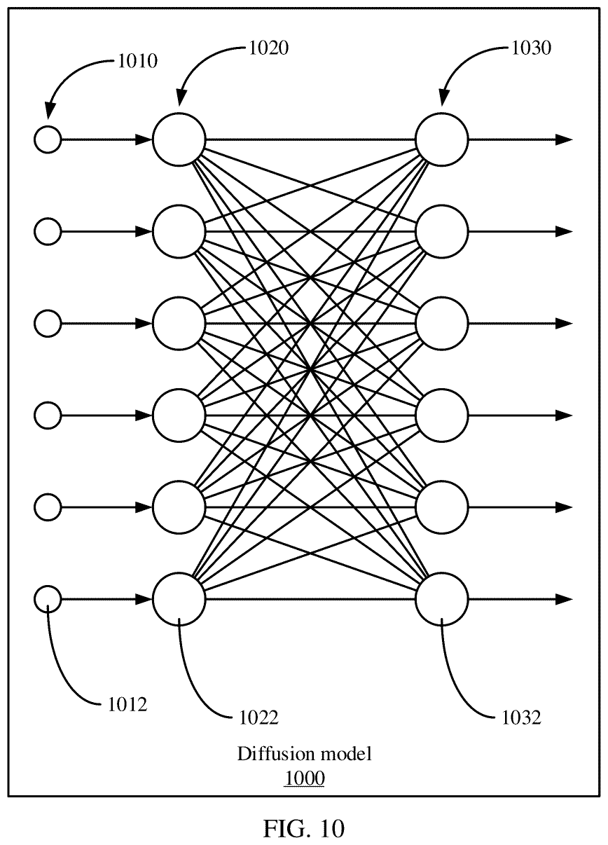

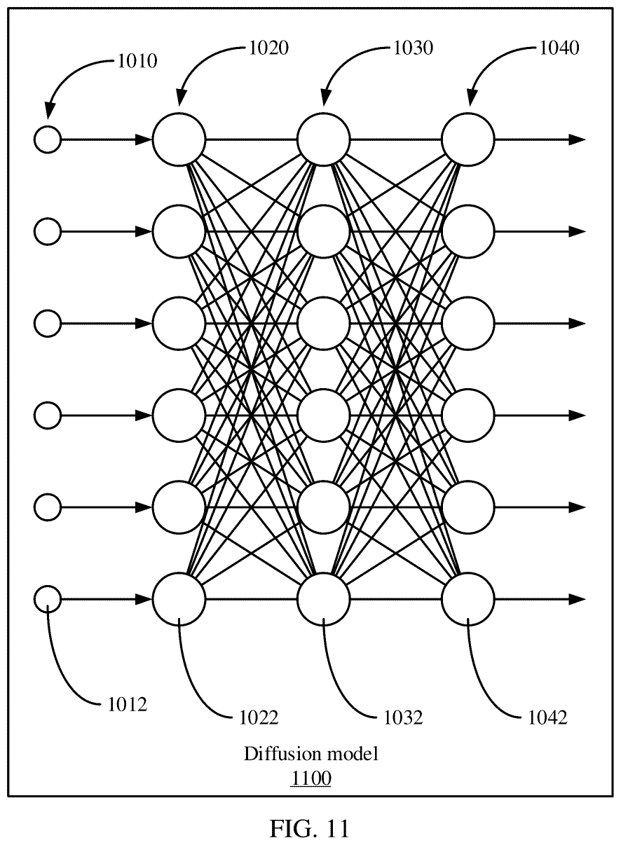

Referring now to FIGS. 10 and 11, exemplary neural network architectures are shown, which may be used to implement parts of the present machine learning models, such as the diffusion model 1000/1100. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 1020 of source nodes 1022, and a single computation layer 1030 having one or more computation nodes 1032 that also act as output nodes, where there is a single computation node 1032 for each possible category into which the input example could be classified. An input layer 1020 can have a number of source nodes 1022 equal to the number of data values 1012 in the input data 1010. The data values 1012 in the input data 1010 can be represented as a column vector. Each computation node 1032 in the computation layer 1030 generates a linear combination of weighted values from the input data 1010 fed into input nodes 1020, and applies a non-linear activation function that is differentiable to the sum. The exemplary simple neural network can perform classification on linearly separable examples (e.g., patterns).

A deep neural network, such as a multilayer perceptron, can have an input layer 1020 of source nodes 1022, one or more computation layer(s) 1030 having one or more computation nodes 1032, and an output layer 1040, where there is a single output node 1042 for each possible category into which the input example could be classified. An input layer 1020 can have a number of source nodes 1022 equal to the number of data values 1012 in the input data 1010. The computation nodes 1032 in the computation layer(s) 1030 can also be referred to as hidden layers, because they are between the source nodes 1022 and output node(s) 1042 and are not directly observed. Each node 1032, 1042 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 1032 in the one or more computation (hidden) layer(s) 1030 perform a nonlinear transformation on the input data 1012 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method, comprising:
recovering an original object from a measured object that includes a distortion, including approximation-free sampling from a posterior distribution of the measured object using a diffusion model; and
performing an action responsive to the recovered original object.

2. The method of claim 1, wherein sampling from the posterior distribution includes sampling from an initial distribution and solving dynamics governing posterior evolution.

3. The method of claim 1, wherein sampling from the posterior distribution includes a stochastic weighted particle approach.

4. The method of claim 1, wherein sampling from the posterior distribution includes solving a stochastic differential equation.

5. The method of claim 4, wherein solving the stochastic differential equation includes resampling to eliminate low-weight particles and duplicate high-weight particles.

6. The method of claim 1, wherein sampling from the posterior distribution includes solving a probability flow ordinary differential equation.

7. The method of claim 6, wherein solving the probability flow ordinary differential equation includes a stochastic corrector to enhance distribution exploration.

8. The method of claim 1, wherein the measured object is an image, the original object is a scene captured in the image, and the distortion includes blurring.

9. The method of claim 1, wherein original object is patient health data and wherein the action includes a treatment action responsive to a health condition of the patient to assist in medical decision making.

10. The method of claim 1, wherein the approximation-free diffusion model is implemented as a machine learning model.

11. A system, comprising:
a hardware processor; and
a memory that stores a computer program which, when executed by the hardware processor, causes the hardware processor to:
recover an original object from a measured object that includes a distortion, including approximation-free sampling from a posterior distribution of the measured object using a model; and
perform an action responsive to the recovered original object.

12. The system of claim 11, wherein sampling from the posterior distribution includes sampling from an initial distribution and solving dynamics governing posterior evolution.

13. The system of claim 11, wherein sampling from the posterior distribution includes a stochastic weighted particle approach.

14. The system of claim 11, wherein sampling from the posterior distribution includes solving a stochastic differential equation.

15. The system of claim 14, wherein solving the stochastic differential equation includes resampling to eliminate low-weight particles and duplicate high-weight particles.

16. The system of claim 11, wherein sampling from the posterior distribution includes solving a probability flow ordinary differential equation.

17. The system of claim 16, wherein solving the probability flow ordinary differential equation includes a stochastic corrector to enhance distribution exploration.

18. The system of claim 11, wherein the measured object is an image, the original object is a scene captured in the image, and the distortion includes blurring.

19. The system of claim 11, wherein original object is patient health data and wherein the action includes a treatment action responsive to a health condition of the patient to assist in medical decision making.

20. The system of claim 11, wherein the approximation-free diffusion model is implemented as a machine learning model.
